(19) United States
(12) Patent Application Publication
(10) Pub. No.: US 2010/0227801 A1
(43) Pub. Date: Sep. 9, 2010

(54) PHARMACEUTICAL COMPOSITIONS

(75) Inventor: Samuel Earl HOPKINS, Research Triangle Park, NC (US)

Correspondence Address: BUCHANAN, INGERSOLL & ROONEY PC, POST OFFICE BOX 1404, ALEXANDRIA, VA, 22313-1404, US

(73) Assignee: Scynexis, Inc., Research Triangle Park, NC (US)

(21) Appl. No.: 12/683,691

(22) Filed: Jan. 7, 2010

Related U.S. Application Data
(60) Provisional application No. 61/143,062, filed on Jan. 7, 2009.
(60) Provisional application No. 61/156,026, filed on Feb. 27, 2009.
(60) Provisional application No. 61/257,231, filed on Nov. 2, 2009.

(51) Int. Cl. A61K 38/13; A61K 31/7072; A61K 31/7068; A61P 31/14

(52) U.S. Cl. 514/11; 514/50; 514/49; 514/51

(57) ABSTRACT

This invention relates to combinations comprising 3-[(R)-2-(N,N-dimethylamino)ethylthio-Sar]-4-(gammahydroxymethylleucine)cyclosporine, or a pharmaceutically acceptable salt, solvate or hydrate thereof; and certain nucleoside analogues, and their use in the treatment of hepatitis C virus.

### 1. CROSS REFERENCE TO RELATED APPLICATIONS

The present application claims the benefit of priority of U.S. Provisional Application Nos. 61/143,062 filed Jan. 7, 2009; 61/156,026 filed Feb. 27, 2009; and 61/257,231 filed Nov. 2, 2009, the contents of which are hereby incorporated by reference in their entireties and relied upon.

### 2. FIELD OF THE INVENTION

The present invention provides methods and pharmaceutical compositions, for use in treatment of hepatitis C virus (HCV) infection in a subject in need thereof.

### 3. BACKGROUND

The current standard of care for treatment of HCV includes interferon and interferon in combination with ribavirin, such as pegylated interferon and ribavirin. A sustained clinical improvement is seen in approximately 50% of patients with genotype I HCV. Thus, the effectiveness of therapy for chronic hepatitis C is low. Moreover, therapy is often associated with considerable side effects. These therapies suffer from a low sustained response rate and frequent side effects (Hoofnagle et al. (1997) N. Engl. J. Med. 336:347).

The emergence of highly active direct antiviral agents, such as HCV protease inhibitors (such as telaprevir and boceprevir) and polymerase inhibitors offers the promise of improving the clinical outcomes in HCV infected patients. However, a patient may become resistant to a particular treatment modality. There have been disclosures of HCV variants with reduced susceptibility to anti-HCV agents. Thus, there is a need for new treatments and therapies for HCV infection. The present invention seeks to provide combinations useful in the treatment or amelioration of one or more symptoms of HCV. The present invention further seeks to provide methods of treatment or amelioration of one or more symptoms of HCV.

3-Substituted ether and thioether cyclosporines and their use to treat or manage certain viral infections are known in the literature. One such compound is SCY-635, which is 3-[(R)-2-(N,N-dimethylamino)ethylthio-Sar]-4-(gammahydroxymethylleucine)cyclosporine. The use of SCY-635 to treat HIV or AIDS is described in U.S. Pat. No. 5,994,299, and its use to treat HCV is described in U.S. Pat. No. 7,196,161. SCY-635 exhibits potent and selective inhibition of HCV-specific RNA replication in both the subgenomic and full length replicon systems in the absence of immunosuppressive activity (Li, K., et al. (2006) “Preclinical evaluation of SCY-635, a cyclophilin inhibitor with potent anti-HCV activity,” Abstract number 934, American Association for the Study of Liver Disease).

In recent year ribonucleoside analogues with a 2′-C-methyl substituent have been identified which possess potent antiviral activity, For example, International Patent Publication Nos. WO2007/065829 and WO2009/121634 describe certain nucleotides that inhibit HCV NS5B polymerase. In particular the nucleoside analogue beta-D-(2′R)-2′-deoxy-2′-fluoro-2′-C-methylcytidine (PSI-6130) has been described as a potent and selective inhibitor of HCV and the diisobutyryl pro-drug of PSI-6130, known as R-7128, is undergoing clinical trials for treating HCV patients. In addition, the nucleoside phosphoramidate pro-drug known as PSI-7851 has demonstrated activity against HCV infected patients in a phase Ib clinical trial (Pharmasset Press Release, Jul. 31, 2009).

Combinations of 3-substituted ether and thioether cyclosporines with other anti-HCV agents, including certain NS5B polymerase inhibitors such as NM-103, an oral pro-drug of NM-107 (2′-C-methyl-cytidine), are described in International Patent Publication No. WO2007/041632.

A need exists for therapies that prevent and/or reduce any adverse or unwanted effects or provide an optimal therapeutic effect or both, that is, provide a desirable therapeutic profile. A need also exists for therapies that allow either the removal or the reduction in the duration of Interferon based treatments. A need also exists for therapies that reduce the risk of viral resistance developing in patients undergoing treatment. A need also exists for an all oral dosage form of therapy, which is a convenient way in which to treat a patient and reduces the risk of non-compliance during therapy.

### 4. SUMMARY OF THE INVENTION

The present invention provides methods of treating HCV infection in a subject in need thereof which comprises administering to the subject a therapeutically effective amount of:

(a) 3-[(R)-2-(N,N-dimethylamino)ethylthio-Sar]-4-(gammahydroxymethylleucine)cyclosporine, or a pharmaceutically acceptable salt, solvate or hydrate thereof; and
(b) a compound of formula (I):

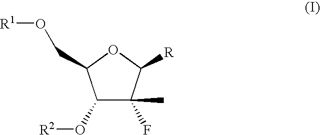

wherein R represents formula (Ia) or (Ib):

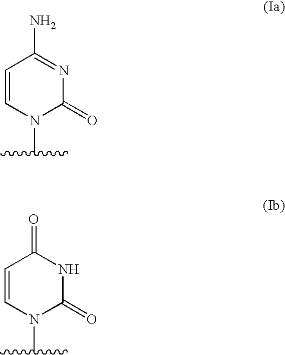

and R¹ and R² independently represent hydrogen, a phosphate-containing group, or a group —C(═O)CH(alkyl)₂;
or a pharmaceutically acceptable hydrate, solvate or an acid addition salt, or pro-drug thereof.

In one aspect, the present invention provides pharmaceutical compositions, single unit dosage forms, and kits suitable for use in treating HCV infection which comprise a therapeutically effective amount of SCY-635 and a compound of formula (I).

In another aspect, the present invention provides pharmaceutical compositions, single unit dosage forms, and kits suitable for use in treating HCV infection which comprise a synergistic effective amount of SCY-635 and a compound of formula (I).

In a further aspect, the present invention provides pharmaceutical compositions, single unit dosage forms, and kits suitable comprising a therapeutically effective amount of SCY-635, a compound of formula (I) and one or more pharmaceutically acceptable carriers or diluents.

### 5. BRIEF DESCRIPTION OF THE FIGURES

FIG. 1 shows a graphical depiction of the anti-HCV synergy volume for a combination of 3-[(R)-2-(N,N-dimethylamino)ethylthio-Sar]-4-(gammahydroxymethylleucine)cyclosporine (also known as SCY-635 and SCY-502635) and beta-D-(2′R)-2′-deoxy-2′-fluoro-2′-C-methylcytidine (PSI-6130) in HCV Replicon ET.

### 6. DESCRIPTION OF EXEMPLARY EMBODIMENTS

The present invention provides methods of treating or preventing hepatitis C infection in a subject in need thereof, and pharmaceutical compositions and dosage forms useful for such methods. The methods and compositions are described in detail in the sections below.

### 6.1 DEFINITIONS

When referring to the compounds and complexes of the invention, the following terms have the following meanings unless indicated otherwise.

“Alkyl” refers to monovalent saturated aliphatic hydrocarbyl groups particularly having up to 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like.

“Aryl” refers to an optionally substituted aromatic hydrocarbon radical, for example phenyl.

“Aralkyl” refers to an aryl group as herein defined attached through an alkylene linker, for example benzyl.

“Cycloalkyl” refers to a saturated, cyclic hydrocarbon moiety having from 3 to 10 ring carbon atoms, more particularly from 3 to 6 ring carbon atoms. This term is exemplified by groups such as cyclopropyl, cyclobutyl, cyclohexyl and the like.

“Nucleosides” are substrate analogs that act as competitive inhibitors of naturally occurring ribonucleoside precursors.

“Pharmaceutically acceptable salt” refers to any salt of a compound of this invention which retains its biological properties and which is not toxic or otherwise undesirable for pharmaceutical use. Such salts may be derived from a variety of organic and inorganic counter-ions well known in the art and include. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethanedisulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylenediamine, chloroprocaine, diethanolamine, procaine, N-benzylphenethylamine, N-methylglucamine piperazine, tris(hydroxymethyl)-aminomethane, tetramethylammonium hydroxide, and the like.

Salts further include, by way of example only, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethanedisulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “physiologically acceptable cation” refers to a non-toxic, physiologically acceptable cationic counterion of an acidic functional group. Such cations are exemplified by sodium, potassium, calcium, magnesium, ammonium and tetraalkylammonium cations and the like.

The term “prodrug” is used throughout the specification to describe any pharmaceutically acceptable form (such as an ester, e.g. an alkyl ester, aralkyl ester, aryl ester; a phosphate ester, a salt of an ester or related group) of a compound which upon administration to a mammal, provides the active compound.

“Solvate” refers to a compound of the present invention or a salt thereof, that further includes a stoichiometric or non-stoichiometric amount of solvent bound by non-covalent intermolecular forces. Where the solvent is water, the solvate is a hydrate.

It is to be understood that compounds having the same molecular formula but differing in the nature or sequence of bonding of their atoms or in the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78:413-447, Angew. Chem., Int. Ed. Engl. 5:385-414 (errata: Angew. Chem., Int. Ed. Engl. 5:511); Prelog and Helmchen, 1982, Angew. Chem. 94:614-631, Angew. Chem. Internat. Ed. Eng. 21:567-583; Mata and Lobo, 1993, Tetrahedron: Asymmetry 4:657-668) or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof. A mixture containing equal proportions of enantiomers is called a “racemic mixture.”

“Sarcosine” or “Sar” refers to the amino acid residue known to those of skill in the art having the structure —N(Me)CH₂C(O)—. Those of skill in the art might recognize sarcosine as N-methyl glycine.

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In one embodiment, the subject is refractory or non-responsive to current treatments for hepatitis C infection. In another embodiment, the subject is a farm animal (e.g., a horse, a cow, a pig, etc.) or a pet (e.g., a dog or a cat). In one embodiment, the subject is a human.

As used herein, the terms “therapeutic agent” and “therapeutic agents” refer to any agent(s) which can be used in the treatment of a disorder or one or more symptoms thereof. In certain embodiments, the term “therapeutic agent” refers to a compound of the invention. In certain other embodiments, the term “therapeutic agent” does not refer to a compound of the invention. In one embodiment, a therapeutic agent is an agent which is known to be useful for, or has been or is currently being used for, the treatment of a disorder or one or more symptoms thereof.

“Therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in one embodiment, to ameliorating a disease or disorder that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” refers to a compound of the invention. In certain other embodiments, the term “prophylactic agent” does not refer a compound of the invention. For example, a prophylactic agent is an agent which is known to be useful for, or has been or is currently being used to prevent or impede the onset, development, progression and/or severity of a disorder.

As used herein, the terms “prevent,” “preventing” and “prevention” refer to the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the phrase “prophylactically effective amount” refers to the amount of a therapy (e.g., prophylactic agent) which is sufficient to result in the prevention of the development, recurrence or onset of one or more symptoms associated with a disorder (, or to enhance or improve the prophylactic effect(s) of another therapy (e.g., another prophylactic agent).

As used herein, the term “in combination” refers to the use of more than one therapy (e.g., one or more prophylactic and/or therapeutic agents). The use of the term “in combination” does not restrict the order in which therapies (e.g., prophylactic and/or therapeutic agents) are administered to a subject with a disorder. A first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disorder.

### 6.2 EMBODIMENTS OF THE INVENTION

The present invention is based, in part, on the discovery that the combinations of the invention are effective for the treatment of hepatitis C infection in a subject in need thereof. Accordingly, the present invention provides methods of treating hepatitis C infection in a subject in need thereof.

While not intending to be bound by any particular theory of operation, it is believed that combinations of the invention inhibit hepatitis C virus (HCV) replication by a mechanism distinct from that of current HCV therapy. Current therapy for HCV, as mentioned above, is co-administration of interferon and ribavirin. It is believed that the current therapy operates by modulation of the immune system of a subject to treat or prevent infection by HCV. It is believed that combinations of the present invention operate by modulating or inhibiting cellular processes critical for HCV replication in a host. Operating by a novel mechanism, the compositions and methods of the invention offer a novel therapy for the treatment of HCV infection. As such they are advantageous for any subject infected with, or at risk for infection with, HCV and particularly for subjects that have not responded to current therapy.

It will be understood that as used herein, reference to amounts of SCY-635 refer to the amount of free base (i.e. 3-[(R)-2-(N,N-dimethylamino)ethylthio-Sar]-4-(gammahydroxymethylleucine)cyclosporine).

In a further embodiment, provided herein are methods for treating or managing HCV infection in a human subject infected with, or at risk for infection with, HCV, the method comprising administering to the human subject a pharmaceutical composition comprising an effective amount of SCY-635, or a pharmaceutically acceptable salt, solvate or hydrate thereof, at least two times in the course of a 24 hour period. In a still further embodiment, the administration is made two or three times per day continually, for a number of days, weeks or months.

In a further embodiment, provided herein are methods of administering SCY-635, or a pharmaceutically acceptable salt, solvate or hydrate thereof, wherein the active agent is administered to an infected human subject in need thereof at least two times in a 24 hour period, wherein each administration is preferably separated by about 4 to about 14 hours.

In another embodiment, provided herein are methods for continual therapy wherein SCY-635, or a pharmaceutically acceptable salt, solvate or hydrate thereof, is administered to an infected human subject in need thereof for a certain period of time (e.g., 5, 7, 10, 14, 20, 24, 28, 60, 120, 360 days or longer).

In another embodiment, provided herein are methods for the administration of SCY-635, or a pharmaceutically acceptable salt, solvate or hydrate thereof, in divided doses (e.g., two or three times daily) of between about 4 mg/kg and about 50 mg/kg; between about 10 mg/kg and about 50 mg/kg; between about 10 mg/kg and about 34 mg/kg; between about 13 mg/kg and about 27 mg/kg; between about 14 mg/kg and about 20 mg/kg; between about 15 mg/kg and about 19 mg/kg; or between about 15 mg/kg and about 18 mg/kg, to a human subject infected with, or at risk for infection with, HCV. In a particular embodiment, SCY-635, or a pharmaceutically acceptable salt, solvate or hydrate thereof, is administered in a dose of about 10 mg/kg, about 13 mg/kg, about 14 mg/kg, about 15 mg/kg, about 17 mg/kg or about 18 mg/kg. In another embodiment, any dose of the SCY-635, or a pharmaceutically acceptable salt, solvate or hydrate, described in the preceding embodiment is administered two or three times in a 24 hour period.

In another embodiment, provided herein are methods for treating a human subject infected with HCV, which include administering to the human subject SCY-635 (a) in an amount of about 200 mg each time, 3 times per day; (b) in an amount of about 250 mg each administration, 3 times per day; (c) in an amount of about 280 mg each administration, 3 times per day; (d) in an amount of about 300 mg each time, 3 times per day; (e) in an amount of about 330 mg each time, 3 times per day; (f) in an amount of about 350 mg each time, 3 times per day; (g) in an amount of about 400 mg each time, 3 times per day; (h) in an amount of about 500 mg each time, 3 times per day; or (i) in an amount of about 600 mg each time, 3 times per day. In one of aspect of the above embodiments, SCY-635 is administered to a human subject once every 8 hours. In another aspect of the above embodiments, SCY-635 is administered at 7-, 7- and 10-hour intervals per day (e.g. at about 7:00 AM, about 2:00 PM, and at about 9:00 PM).

In another embodiment, provided herein are methods for treating a human subject infected with HCV, which include administering to the human subject SCY-635 (a) in an amount of about 300 mg each time, 2 times per day, once every 12 hours; (b) in an amount of about 400 mg each administration, 2 times per day, once every 12 hours; (c) in an amount of about 425 mg each administration, 2 times per day, once every 12 hours; (d) in an amount of about 450 mg each time, 2 times per day, once every 12 hours; (e) in an amount of about 500 mg each time, 2 times per day, once every 12 hours; (f) in an amount of about 550 mg each time, 2 times per day, once every 12 hours; (g) in an amount of about 600 mg each time, 2 times per day, once every 12 hours; (h) in an amount of about 625 mg each time, 2 times per day, once every 12 hours; (i) in an amount of about 650 mg each time, 2 times per day, once every 12 hours; (j) in an amount of about 700 mg each time, 2 times per day, once every 12 hours; or (k) in an amount of about 800 mg each time, 2 times per day, once every 12 hours.

A method that provides greater than 600 mg of SCY-635, given as a divided dose over a 24 hour period, can effectively result in a high trough level of SCY-635 in plasma. As used herein, “trough level” refers to the lowest level that a medicine is present in the body. It can be important, particularly in viral diseases, to maintain drug levels above a certain concentration to maintain appropriate inhibition of viral replication. In particular, it has been found that a dosing regimen of greater than about 200 mg of SCY-635 each time, three times a day, once every 8 hours, can lead to disproportionately higher trough levels of SCY-635 than seen at lower concentrations. It will be understood however that in the presence of a synergistic second agent, such as a compound of formula (I) the dose of SCY-635 may be lower, e.g. 400 mg over a 24 hour period.

In another embodiment, provided herein are methods for treating a human subject infected with HCV, given as a divided dose over a 24 hour period, which include administering to the human subject SCY-635 (a) in an amount of from 800 to 999 mg per day; (b) in an amount of from 810 to 997 mg per day; (c) in an amount of from 820 to 995 mg per day; (d) in an amount of from 850 to 950 mg per day; (e) in an amount of 870 to 930 mg per day; (f) in an amount of from 880 to 920 mg per day; or (g) in an amount of from 890 to 910 mg per day. In one aspect of these embodiments SCY-635 is given in two doses over a 24 hour period. In another aspect of these embodiments SCY-635 is given in three doses over a 24 hour period.

In another embodiment, provided herein are methods for treating a human subject infected with HCV, which include administering to the human subject SCY-635, given as a divided dose over a 24 hour period, (a) in an amount of at least 1001 mg per day; (b) in an amount of at least 1003 mg per day; (c) in an amount of at least 1005 mg per day; (d) in an amount of from 1010 to 1200 mg per day; (e) in an amount of from 1020 to 1200 mg per day; (e) in an amount of 1040 to 1150 mg per day; (f) in an amount of from 1050 to 1120 mg per day; or (g) in an amount of from 1060 to 1100 mg per day. In one aspect of these embodiments SCY-635 is given in two doses over a 24 hour period. In another aspect of these embodiments SCY-635 is given in three doses over a 24 hour period.

In another embodiment, a therapeutically effective plasma concentration of SCY-635 is obtained and a certain trough level concentration of SCY-635 is maintained at steady state. These methods can be particularly useful for treating a human infected with HCV by administering an SCY-635 formulation, wherein a trough SCY-635 plasma level is maintained at a minimum of about 110 ng/mL, about 115 ng/mL, about 135 ng/mL, about 216 ng/mL, or about 400 ng/mL, over a 24 hour period at steady state. In certain embodiments, the methods can be particularly useful for treating a human suffering from HCV infection by administering an SCY-635 formulation, wherein the trough SCY-635 plasma level is maintained at a minimum of about 115 ng/mL over a 24 hour period at steady state. In certain embodiments, the methods can be particularly useful for treating or managing HCV infection in a human subject infected with, or at risk for infection with, HCV, wherein the compound is administered in amount sufficient to maintain a trough plasma concentration of the compound of greater than about 115 ng/ml at steady state.

In one embodiment the compound of formula (I) is administered in an amount from about 50 mg per day to about 5000 mg per day. In another embodiment the compound of formula (I) is administered in an amount from about 100 mg per day to about 3000 mg per day.

In one embodiment of the invention the compound of formula (I) is a compound wherein R is formula (Ia) and R¹ and R² each represent hydrogen, known as PSI-6130. In another embodiment of the invention the compound of formula (I) is a compound wherein R is formula (Ia) and R¹ and R² each represent hydrogen or R¹ and R² each represent —C(═O)CH(CH₃)₂, known as R-7128. In a further embodiment of the invention the compound of formula (I) is a compound wherein R is formula (Ib), R² is hydrogen and R¹ is a phosphoramidate, e.g. of formula —P(═O)(OR³)NHCH(R⁴)CO₂R⁵ in which R³ represents hydrogen, alkyl, cycloalkyl or aryl; R⁴ is hydrogen or alkyl; and R⁵ is hydrogen or alkyl. In a still further embodiment of the invention the compound of formula (I) is a compound wherein R is formula (Ib), R² is hydrogen and R¹ is the phosphate of formula —P(═O)(OR³)NHCH(R⁴)CO₂R⁵, in which R³ represents phenyl; R⁴ is methyl; and R⁵ is isopropyl, known as PSI-7851. In a further embodiment of the invention the compound of formula (I) is a compound wherein R is formula (Ib), R² is hydrogen and R¹ is the phosphate of formula —P(═O)(OH)—O—P(═O)(OH)—O—P(═O)OH, known as PSI-7409, or a prodrug thereof.

In one embodiment the invention provides administering to a subject

(a) SCY-635 in an amount of from about 800 mg to about 1400 mg per day; and
(b) R-7128 in an amount of from about 800 mg to about 2200 mg per day.

In another embodiment the invention provides administering to a subject

(a) SCY-635 in an amount of from about 900 mg to about 1200 mg per day; and
(b) R-7128 in an amount of from about 800 mg to about 2200 mg per day.

In one embodiment the invention provides administering to a subject

(a) SCY-635 in an amount of from about 400 mg to about 1000 mg per day; and
(b) R-7128 in an amount of from about 800 mg to about 2200 mg per day.

In another embodiment the invention provides administering to a subject

(a) SCY-635 in an amount of from about 800 mg to about 1000 mg per day; and
(b) R-7128 in an amount of from about 800 mg to about 2200 mg per day.

In embodiments of the invention, the subject can be any subject infected with, or at risk for infection with, HCV. Infection or risk for infection can be determined according to any technique deemed suitable by the practitioner of skill in the art. In one embodiment, subjects are humans infected with HCV.

In one embodiment of the invention the combination of (a) and (b) is administered to a subject for from about 2 weeks to about 96 weeks. In another embodiment of the invention, the combination of (a) and (b) is administered for from about 6 weeks to about 72 weeks. In a still further embodiment of the invention, the combination of (a) and (b) is administered for from about 12 weeks to about 48 weeks. In a still further embodiment of the invention, the combination of (a) and (b) is administered for from about 12 weeks to about 24 weeks.

The HCV can be any HCV known to those of skill in the art. There are at least six genotypes and at least 50 subtypes of HCV currently known to those of skill in the art. The HCV can be of any genotype or subtype known to those of skill. In certain embodiments, the HCV is of a genotype or subtype not yet characterized. In certain embodiments, the subject is infected with HCV of a single genotype. In certain embodiments, the subject is infected with HCV of multiple subtypes, quasispecies, or multiple genotypes.

In certain embodiments, the HCV is genotype 1 and can be of any subtype. For instance, in certain embodiments, the HCV is subtype 1a, 1b or 1c. It is believed that HCV infection of genotype 1 responds poorly to current interferon therapy. Methods of the present invention can be advantageous for therapy of HCV infection with genotype 1.

In certain embodiments, the HCV is other than genotype 1. In certain embodiments, the HCV is genotype 2 and can be of any subtype. For instance, in certain embodiments, the HCV is subtype 2a, 2b or 2c. In certain embodiments, the HCV is genotype 3 and can be of any subtype. For instance, in certain embodiments, the HCV is subtype 3a, 3b or 10a. In certain embodiments, the HCV is genotype 4 and can be of any subtype. For instance, in certain embodiments, the HCV is subtype 4a. In certain embodiments, the HCV is genotype 5 and can be of any subtype. For instance, in certain embodiments, the HCV is subtype 5a. In certain embodiments, the HCV is genotype 6 and can be of any subtype. In certain embodiments, the HCV is subtype 6a, 6b, 7b, 8b, 9a or 11a. See, e.g., Simmonds, 2004, J Gen Virol. 85:3173-88; Simmonds, 2001, J. Gen. Virol., 82, 693-712, the contents of which are incorporated by reference in their entirety.

In certain embodiments of the invention, the subject has never received therapy or prophylaxis for HCV infection. In further embodiments of the invention, the subject has previously received therapy or prophylaxis for HCV infection. For instance, in certain embodiments, the subject has not responded to HCV therapy. Indeed, under current interferon therapy, up to 50% or more HCV subjects do not respond to therapy. In certain embodiments, the subject can be a subject that received therapy but continued to suffer from viral infection or one or more symptoms thereof. In certain embodiments, the subject can be a subject that received therapy but failed to achieve a sustained virologic response. In certain embodiments, the subject has received therapy for HCV infection but has failed show a 2 log₁₀ decline in HCV RNA levels after 12 weeks of therapy. It is believed that subjects who have not shown more than 2 log₁₀ reduction in serum HCV RNA after 12 weeks of therapy have a 97-100% chance of not responding. Since the combinations of the present invention act by mechanism other than current HCV therapy, it is believed that combinations of the invention should be effective in treating such nonresponders.

In certain embodiments, the subject is a subject that discontinued HCV therapy because of one or more adverse events associated with the therapy. In certain embodiments, the subject is a subject where current therapy is not indicated. For instance, certain therapies for HCV are associated with neuropsychiatric events. Interferon (IFN)-alpha plus ribavirin is associated with a high rate of depression. Depressive symptoms have been linked to a worse outcome in a number of medical disorders. Life-threatening or fatal neuropsychiatric events, including suicide, suicidal and homicidal ideation, depression, relapse of drug addiction/overdose, and aggressive behavior have occurred in subjects with and without a previous psychiatric disorder during HCV therapy. Interferon-induced depression is a limitation for the treatment of chronic hepatitis C, especially for subjects with psychiatric disorders. Psychiatric side effects are common with interferon therapy and responsible for about 10% to 20% of discontinuations of current therapy for HCV infection.

Accordingly, the present invention provides methods of treating HCV infection in subjects where the risk of neuropsychiatric events, such as depression, contraindicates treatment with current HCV therapy. The present invention also provides methods of treating or preventing HCV infection in subjects where a neuropsychiatric event, such as depression, or risk of such indicates discontinuation of treatment with current HCV therapy. The present invention further provides methods of treating or preventing HCV infection in subjects where a neuropsychiatric event, such as depression, or risk of such indicates dose reduction of current HCV therapy.

Current therapy is also contraindicated in subjects that are hypersensitive to interferon or ribavirin, or both, or any other component of a pharmaceutical product for administration of interferon or ribavirin. Current therapy is not indicated in subjects with hemoglobinopathies (e.g., thalassemia major, sickle-cell anemia) and other subjects at risk from the hematologic side effects of current therapy. Common hematologic side effects include bone marrow suppression, neutropenia and thrombocytopenia. Furthermore, ribavirin is toxic to red blood cells and is associated with hemolysis. Accordingly, the present invention also provides methods of treating HCV infection in subjects hypersensitive to interferon or ribavirin, or both, subjects with a hemoglobinopathy, for instance thalassemia major subjects and sickle-cell anemia subjects, and other subjects at risk from the hematologic side effects of current therapy.

In certain embodiments, the subject has received HCV therapy and discontinued that therapy prior to administration of a method of the invention. In further embodiments, the subject has received therapy and continues to receive that therapy along with administration of a method of the invention. The methods of the invention can be co-administered with other therapy for HCV according to the judgment of one of skill in the art. In certain embodiments, the methods or compositions of the invention can be co-administered with a reduced dose of the other therapy for HCV.

In certain embodiments, the present invention provides methods of treating a subject that is refractory to treatment with interferon. For instance, in some embodiments, the subject can be a subject that has failed to respond to treatment with one or more agents selected from the group consisting of interferon, interferon-alpha, pegylated interferon α, interferon plus ribavirin, interferon α plus ribavirin and pegylated interferon α plus ribavirin. In some embodiments, the subject can be a subject that has responded poorly to treatment with one or more agents selected from the group consisting of interferon, interferon α, pegylated interferon α, interferon plus ribavirin, interferon α plus ribavirin and pegylated interferon α plus ribavirin. In the above embodiments, a pro-drug form of ribavirin, such as taribavirin, may also be used.

In further embodiments, the present invention provides methods of treating HCV infection in subjects that are pregnant or might get pregnant since current therapy is also contraindicated in pregnant women.

In certain embodiments, the subject has, or is at risk for, co-infection of HCV with HIV. For instance, in the United States, 30% of HIV subjects are co-infected with HCV and evidence indicates that people infected with HIV have a much more rapid course of their hepatitis C infection. Maier and Wu, 2002, World J Gastroenterol 8:577-57. The methods of the invention can be used to treat HCV infection in such subjects. It is believed that elimination of HCV in these subjects will lower mortality due to end-stage liver disease. Indeed, the risk of progressive liver disease is higher in subjects with severe AIDS-defining immunodeficiency than in those without. See, e.g., Lesens et al., 1999, J Infect Dis 179:1254-1258.

In certain embodiments, the methods or compositions of the invention are administered to a subject following liver transplant. Hepatitis C is a leading cause of liver transplantation in the U.S, and many subjects that undergo liver transplantation remain HCV positive following transplantation. The present invention provides methods of treating such recurrent HCV subjects with a compound or composition of the invention. In certain embodiments, the present invention provides methods of treating a subject before, during or following liver transplant to prevent recurrent HCV infection.

### Pharmaceutical Compositions and Unit Dosage Formulations

Pharmaceutical compositions and single unit dosage forms comprising SCY-635, or a pharmaceutically acceptable salt, solvate or hydrate thereof, and a compound of formula (I), are also provided herein. Individual dosage forms may be suitable for oral, mucosal (including sublingual, buccal, rectal, nasal, or vaginal) or parenteral (including subcutaneous, intramuscular, bolus injection, intraarterial, or intravenous) administration. Preferred pharmaceutical compositions and single unit dosage forms are suitable for oral administration.

In one embodiment, the pharmaceutical composition is a solid oral dosage form. In one embodiment, the pharmaceutical composition is a liquid oral dosage form. In a particular embodiment, provided herein are doses, unit dosage formulations and pharmaceutical compositions wherein SCY-635, or a pharmaceutically acceptable salt, solvate or hydrate thereof, and the compound of formula (I), are orally bioavailable. Advantages of oral administration can include ease of administration, higher human subject compliance with the dosing regimen, clinical efficacy, fewer complications, shorter hospital stays, and overall cost savings.

In another embodiment, provided herein are unit dosage formulations that comprise between about 30 mg and about 1400 mg, between about 100 mg and about 1000 mg, between about 200 mg and about 1000 mg, or between about 250 mg and about 1000 mg of SCY-635, or a pharmaceutically acceptable salt, solvate or hydrate thereof; and between about 100 mg and about 3000 mg of the compound of formula (I). In one embodiment, the unit dosage formulation comprises SCY-635, or a pharmaceutically acceptable salt, solvate or hydrate thereof, the compound of formula (I), and one or more carriers or excipients suitable for suspension in a pharmaceutically acceptable solvent (e.g., water, milk, a carbonated beverage, juice, apple sauce, baby food or baby formula) in a bottle.

In another embodiment, provided herein are unit dosage formulations that comprise about 35 mg, about 50 mg, about 70 mg, about 100 mg, about 125 mg, about 140 mg, about 175 mg, about 200 mg, about 250 mg, about 280 mg, about 350 mg, about 500 mg, about 560 mg, about 700 mg, about 750 mg, about 1000 mg or about 1400 mg of SCY-635, or a pharmaceutically acceptable salt, solvate or hydrate thereof. Preferred unit dosage formulations comprise about 125 mg, about 250, about 300 mg, about 500 mg, or about 1000 mg of SCY-635, or a pharmaceutically acceptable salt, solvate or hydrate thereof. In one embodiment, the unit dosage formulation comprises SCY-635, or a pharmaceutically acceptable salt, solvate or hydrate thereof, and one or more carriers or excipients suitable for suspension in a pharmaceutically acceptable solvent (e.g., water, milk, a carbonated beverage, juice, apple sauce, baby food or baby formula) in a bottle. Preferred unit dosage formulations are capsules, powders and sachets. A particularly preferred unit dosage is a capsule.

Single unit dosage forms suitable for oral administration to a human subject include, but are not limited to: sachets; cachets; tablets; caplets; capsules, such as soft elastic gelatin capsules; troches; lozenges; dispersions; powders; solutions; liquid dosage forms, including suspensions (e.g., aqueous or non-aqueous liquid suspensions); emulsions (e.g., oil-in-water emulsions, or a water-in-oil liquid emulsion); and elixirs. In one embodiment, provided herein is a colloid solution or a solution with additional active agent, above the saturating concentration. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). See also Remington: The Science and Practice of Pharmacy, 21^{st} ed., Lippincott Williams & Wilkins, Philadelphia, Pa. (2005)

In another embodiment, provided herein are anhydrous pharmaceutical compositions and dosage forms comprising SCY-635, or a pharmaceutically acceptable salt, solvate or hydrate thereof; and a compound of formula (I). Anhydrous pharmaceutical compositions and dosage forms of the invention can be prepared using anhydrous or low moisture containing ingredients and low moisture or low humidity conditions.

Typical oral dosage forms of the invention are prepared by combining the active ingredient(s) in an intimate admixture with at least one carrier or excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents (e.g., vanilla extract), preservatives, and coloring agents. Examples of excipients suitable for use in solid oral dosage forms (e.g., powders, tablets, sachets, capsules, and caplets) include, but are not limited to, starches, sugars, micro-crystalline cellulose, diluents, granulating agents, lubricants, binders, and disintegrating agents.

In one embodiment, the unit dosage formulations are powder formulations comprising an effective amount of the active agent which are suitable for reconstitution in a pharmaceutically acceptable solvent (e.g., water, milk, a carbonated beverage, juice, apple sauce, baby food or baby formula) and subsequent oral administration. In a particular embodiment, the powder can optionally contain one or more carriers or excipients in combination with the active agent. In another embodiment, the powder can be stored in a sealed container prior to administration or reconstitution. In yet another embodiment, the powder can be encapsulated (e.g., in a gelatin capsule).

The following Examples illustrate the synthesis of representative compounds and their use in the methods of the present invention. These examples are not intended, nor are they to be construed, as limiting the scope of the invention. It will be clear that the invention may be practiced otherwise than as particularly described herein. Numerous modifications and variations of the present invention are possible in view of the teachings herein and, therefore, are within the scope of the invention.

### 7. EXAMPLES

3-[(R)-2-(N,N-dimethylamino)ethylthio-Sar]-4-(gammahydroxymethylleucine)cyclosporine (SCY-502635) was prepared as described in U.S. Pat. No. 5,994,299. Beta-D-(2′R)-2′-deoxy-2′-fluoro-2′-C-methylcytidine (PSI-6130) was prepared using the methods described by Wang et. Al., J. Org. Chem. 2009, 74, 6819.

### HCV Replicon Cell Line.

The ET cell line was provided by Dr. Ralf Bartenschlager at the University of Heidelberg. The ET cell line is a human hepatoma cell line (Huh-7) that contains a con1 (genotype 1b) bi-cistronic subgenomic replicon. The replicon contains a stable luciferase reporter gene and three cell culture adaptive mutations. The subgenomic replicon contains the HCV Internal Ribosomal Entry Site (IRES) and the first few amino acid codons of the HCV core protein which drives the production of luciferase, ubiquitin, and neomycin phosphotransferase fusion protein. The encephalomyocarditis virus (EMCV) IRES element directs the translation of the second cistronic unit that encodes the non-structural proteins NS3, NS4A, NS4B, NS5A, and NS5B. Cells were grown in Dulbecco's modified essential media (DMEM) with 10% fetal bovine serum (FBS), 1% penicillin-streptomycin, 1% glutamine, 1% non-essential amino acids, and 5 mg/ml G418 in a 5% CO₂ incubator at 37° C. All cell culture reagents were obtained from Mediatech (Herndon, Va.).

Antiviral activity and cytotoxicity assay. Assessments of antiviral activity and cytotoxicity were carried out in parallel in clear-bottomed 96-well plates. Recombinant interferon alpha-2b (rIFNα-2b) was included as a positive control at half-log 10 dilutions typically spanning the range of 0.0064 to 2.0 IU/ml. The reporter replicon cells were seeded at a density of 5×103 cells per well in 0.1 ml of DMEM without selection antibiotics in a humidified atmosphere supplemented with 5% CO2 at 37° C. Half-log 10 serial dilutions of the test compounds typically spanning the range of 0.03 to 3.0 μM were prepared in DMEM and then applied to corresponding wells. The cells were processed 72 hours after the incubation. Antiviral activity was assessed as the replicon-derived luciferase activity. Cytotoxicity was assessed by using the CytoTox-One Homogeneous Membrane Integrity Assay Kit (cell proliferation assay, Promega, Madison, Wis.). The percent inhibition was plotted against the nominal concentration of compound to derive values for 50% inhibition of viral replication (EC₅₀) and 50% cell viability (CC).

Drug Combination Assays.

The HCV subgenomic replicons described above, were used to assess the effect of SCY-502635 in combination with PSI-6130. The cells were plated at 5000 cells/well. Plates for antiviral activity and cytotoxicity were prepared in parallel. The following day, test articles were diluted and added to the plates to create 40 to 45 discreet 2-drug combinations. SCY-502635 was tested at eight to nine 2-fold concentrations and the second test article was tested at five 2-fold dilutions. After a 72 hour incubation period, cells were processed to determine antiviral activity (luciferase) or cytotoxicity (LDH release).

To determine whether the effects of the 2-drug combinations were either synergistic, additive, or antagonistic the anti-viral activity data were analyzed using the Prichard and Shipman MacSynergy II data analysis program. In general, statistically significant differences are achieved for any discreet 2-drug combination if the absolute value of the difference between the observed and expected results exceeds the corresponding standard deviation of the observed results by a factor 1.96. The results of the analysis are presented in a 3-dimensional Cartesian coordinate system to yield surfaces of activity that can fall above (indicating synergy), below (indicating antagonism) or in the plane of the central x y axis (indicating additive interactions). A surface volume is calculated with the dimensions of (concentration; x)(concentration; y)(% inhibition; z). Calculated volumes greater than 50 indicate synergy; volumes ranging from −50 to +50 indicate additive effects; volumes less than −50 indicate antagonism. The results of this analysis at the 95% confidence interval are indicative of statistically significant effects.

The results obtained were as follows:

| Antiviral Efficacy | | Cytotoxicity | |
|---|---|---|---|
| Synergy volume | Interpretation | Synergy volume | Interpretation |
| 99.38/0 μM² % | Slightly | 0/−1.53 μM² % | Additive |
| | synergistic | | |
| 112.48/0 μM² % | Highly | 0/−7.64 μM² % | Additive |
| | synergistic | | |
| 76.18/0 μM² % | Slightly | 2.8/−11.01 μM² % | Additive |
| | synergistic | | |

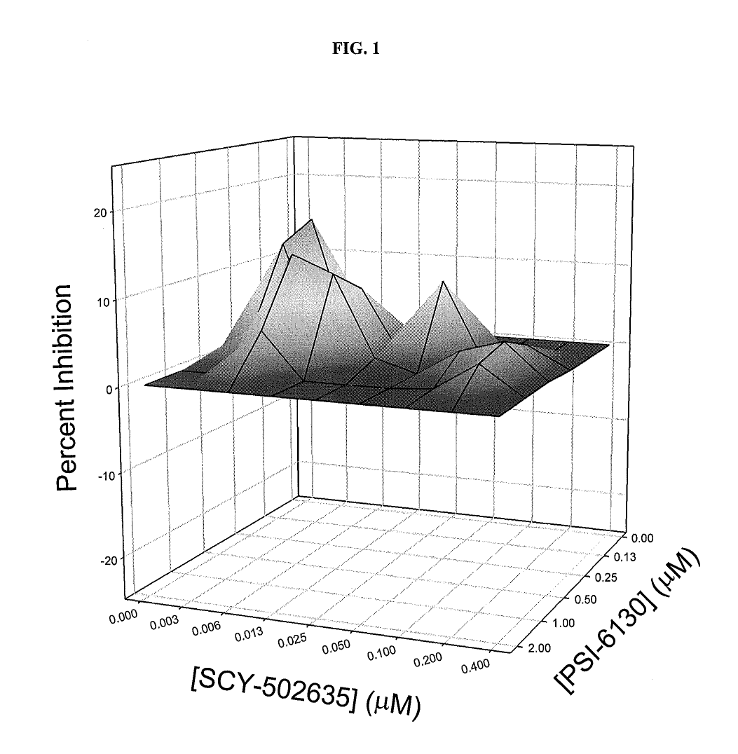

As shown in the table above and FIG. 1, for the combination of SCY-502635 and PSI-6130, at a 95% confidence interval, the antiviral synergy volume was slightly or highly synergistic. As shown in the table above there was no antagonistic cytotoxicity for this combination.

All publications, patents and patent applications cited in this specification are herein incorporated by reference as if each individual publication, patent or patent application were specifically and individually indicated to be incorporated by reference. While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What we claim is:

1. A method for treating HCV infection in a subject in need thereof, which comprises administering to the subject a therapeutically effective amount of:
(a) 3-[(R)-2-(N,N-dimethylamino)ethylthio-Sar]-4-(gammahydroxymethylleucine)cyclosporine, or a pharmaceutically acceptable salt, solvate or hydrate thereof; and
(b) a compound of formula (I): 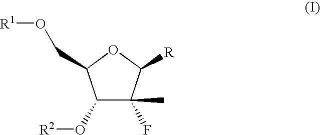
wherein R represents formula (Ia) or (Ib): 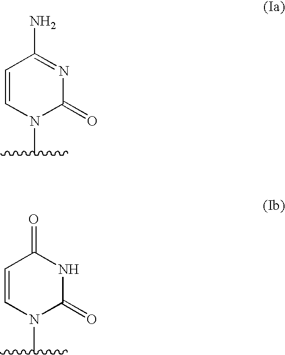
and R¹ and R² independently represent hydrogen, a phosphate-containing group, or a group —C(═O)CH(alkyl)₂;
or a pharmaceutically acceptable hydrate, solvate, acid addition salt or pro-drug thereof.

2. The method according to claim 1, in which the compound of formula (I) is a compound wherein R is formula (Ia) and R¹ and R² each represent hydrogen, or a pro-drug thereof.

3. The method according to claim 1, in which the compound of formula (I) is a compound wherein R is formula (Ia) and R¹ and R² each represent —C(═O)CH(CH₃)₂, known as R-7128.

4. The method according to claim 1, in which the compound of formula (I) is a compound wherein R is formula (Ib), R² is hydrogen and R¹ is a —P(═O)(OH)—O—P(═O)(OH)—O—P(═O)OH, or a pro-drug thereof.

5. The method according to claim 1, in which the compound of formula (I) is a compound wherein R is formula (Ib), R² is hydrogen and R¹ is the phosphate of formula —P(═O)(OR³)NHCH(R⁴)CO₂R⁵, in which R³ represents phenyl; R⁴ is methyl; and R⁵ is isopropyl, known as PSI-7851.

6. The method according to claim 1, in which (a) 400 mg or more of 3-[(R)-2-(N,N-dimethylamino)ethylthio-Sar]-4-(gammahydroxymethylleucine)cyclosporine is administered per day; and (b) the compound of formula (I) is administered in an amount from about 50 mg per day to about 5000 mg per day.

7. A composition comprising:
(a) 3-[(R)-2-(N,N-dimethylamino)ethylthio-Sar]-4-(gammahydroxymethylleucine)cyclosporine, or a pharmaceutically acceptable salt, solvate or hydrate thereof;
(b) a compound of formula (I): 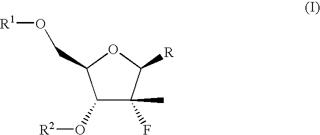
wherein R represents formula (Ia) or (Ib): 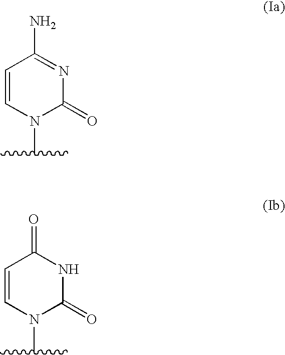
and R¹ and R² independently represent hydrogen, a phosphate-containing group, or a group —C(═O)CH(alkyl)₂;
or a pharmaceutically acceptable hydrate, solvate, acid addition salt or pro-drug thereof;
and a pharmaceutically acceptable carrier or diluent.

8. The composition according to claim 7, in which the compound of formula (I) is a compound wherein R is formula (Ia) and R¹ and R² each represent hydrogen, or a pro-drug thereof.

9. The composition according to claim 7, in which the compound of formula (I) is a compound wherein R is formula (Ia) and R¹ and R² each represent hydrogen or —C(═O)CH(CH₃)₂, known as R-7128.

10. The composition according to claim 7, in which the compound of formula (I) is a compound R is formula (Ib), R² is hydrogen and R¹ is the phosphate of formula —P(═O)(OR³)NHCH(R⁴)CO₂R⁵, in which R³ represents phenyl; R⁴ is methyl; and R⁵ is isopropyl, known as PSI-7851.

11. The composition according to claim 7, in which the compound of formula (I) is a compound wherein R is formula (Ib), R² is hydrogen and R¹ is —P(═O)(OH)—O—P(═O)(OH)—O—P(═O)OH, or a pro-drug thereof.

12. The composition according to claim 7, in which the compound of formula (I) is a compound wherein R is formula (Ib), R² is hydrogen and R¹ is —P(═O)(OH)—O—P(═O)(OH)—O—P(═O)OH, or a pro-drug thereof.

13. A method for treating HCV infection in a subject in need thereof, which comprises administering to the subject a synergistic therapeutically effective amount of:
(a) 3-[(R)-2-(N,N-dimethylamino)ethylthio-Sar]-4-(gammahydroxymethylleucine)cyclosporine, or a pharmaceutically acceptable salt, solvate or hydrate thereof; and
(b) a compound of formula (I): 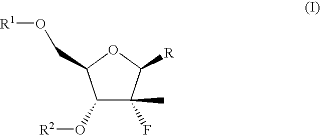
wherein R represents formula (Ia) or (Ib): 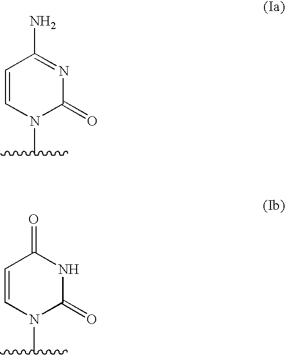
and R¹ and R² independently represent hydrogen, a phosphate-containing group, or a group —C(═O)CH(alkyl)₂;
or a pharmaceutically acceptable hydrate, solvate, acid addition salt or pro-drug thereof.

14. The method according to claim 13, in which the compound of formula (I) is a compound wherein R is formula (Ia) and R¹ and R² each represent hydrogen, or a pro-drug thereof.

15. The method according to claim 13, in which the compound of formula (I) is a compound wherein R is formula (Ia) and R¹ and R² each represent —C(═O)CH(CH₃)₂, known as R-7128.

16. The method according to claim 13, in which the compound of formula (I) is a compound wherein R is formula (Ib), R² is hydrogen and R¹ is a —P(═O)(OH)—O—P(═O)(OH)—O—P(═O)OH, or a pro-drug thereof.

17. The method according to claim 13, in which the compound of formula (I) is a compound wherein R is formula (Ib), R² is hydrogen and R¹ is the phosphate of formula —P(═O)(OR³)NHCH(R⁴)CO₂R⁵, in which R³ represents phenyl; R⁴ is methyl; and R⁵ is isopropyl, known as PSI-7851.

18. The method according to claim 13, comprising administering to the subject (a) 400 mg or more per day of 3-[(R)-2-(N,N-dimethylamino)ethylthio-Sar]-4-(gammahydroxymethylleucine)cyclosporine; and (b) from about 50 mg to about 5000 mg per day of the compound of formula (I).

19. The method according to claim 15, comprising administering to the subject:
(a) 3-[(R)-2-(N,N-dimethylamino)ethylthio-Sar]-4-(gammahydroxymethylleucine)cyclosporine in an amount from about 800 mg to about 1400 mg per day; and
(b) R-7128 in an amount from about 800 mg to about 2200 mg per day.
